(19) United States
(12) Patent Application Publication
(10) Pub. No.: US 2022/0273538 A1
(43) Pub. Date: Sep. 1, 2022

(54) SKIN CARE COMPOSITION INCLUDING BAKUCHIOL AND HEMP OIL

(71) Applicant: Edgewell Personal Care Brands, LLC, Chesterfield, MO (US)

(72) Inventors: Jamie Otlik, Dallas, TX (US); Meghan Montgomery, Huntington, VT (US)

(21) Appl. No.: 17/680,999

(22) Filed: Feb. 25, 2022

Related U.S. Application Data
(60) Provisional application No. 63/154,130, filed on Feb. 26, 2021.

(51) Int. Cl. A61K 8/34; A61K 8/92; A61K 8/06; A61Q 19/08

(52) U.S. Cl. CPC A61K 8/347; A61K 8/922; A61K 8/06; A61Q 19/08; A61K 2800/30; A61K 2800/87

(57) ABSTRACT

A skin care composition includes a skin sensory enhancer including bakuchiol, an oil-based skin conditioning agent including hemp oil, and optionally one or more additional ingredients. A method to provide skin sensory enhancement and/or moisturization includes contacting skin with the skin care composition. A method to make the skin care composition includes mixing at least the skin sensory enhancer, including the bakuchiol, with the oil-based skin conditioning agent, including the Cannabis sativa seed oil. In one example, the skin care composition is in the form of an emulsion and is applied from a pump container.

### CROSS-REFERENCE TO RELATED APPLICATIONS

This application claims priority to U.S. Provisional Patent Application No. 63/154,130, filed on Feb. 26, 2021, the contents of which are incorporated herein by reference in their entirety.

### TECHNICAL FIELD

Embodiments relate to skin care compositions including bakuchiol and hemp oil.

### BACKGROUND

Skin care compositions may include a variety of ingredients for anti-aging purposes. For example, retinol may boost an amount of collagen, minimize fine lines or wrinkles, and improve skin tone or color. Such ingredients may, however, increase skin irritation. Thus, there is considerable room for improvement to provide topical compositions having skin benefits with substantially good sensory profiles such as relatively low irritation, relatively good moisturization, and/or relatively good soothing properties.

### SUMMARY

Embodiments relate to a skin care composition including a skin sensory enhancer and a skin conditioning agent. The skin sensory enhancer preferably includes bakuchiol, which may be present in an amount of about 0.25 wt. % to about 5.0 wt. %, based on the total weight of the skin care composition. The skin conditioning agent preferably includes hemp oil (hemp seed oil, Cannabis sativa seed oil, etc.), which may be present in an amount of about 0.1 wt. % to about 3.0 wt. %, based on the total weight of the skin care composition, and one or more additional ingredients. In one example, the skin care composition is free of retinol.

Embodiments relate to a method to provide skin sensory enhancement. In one example, the skin sensory enhancement may include relative improved smoothness of skin, improved overall feel of skin, brighter-looking skin, firmer skin, improved overall appearance of skin, improved overall texture of skin, reduced signs of aging, reduced appearance of fine lines, reduced appearance of wrinkles, or combinations thereof. The skin sensory enhancement may include, for example, relatively fewer fine lines. The skin care composition may further provide moisturization to the skin. In one example, the moisturization may include relative improved hydration. The moisturization may minimize indications of irritations such as, for example, dryness, edema, erythema, or combinations thereof.

The method may include contacting skin with the skin care composition. In one example, contacting the skin with the skin care composition may include applying the skin care composition to the skin at any time throughout a day. In one example, contacting the skin with the skin care composition may include applying the skin care composition to the skin at bedtime. Additionally or alternatively, contacting the skin with the skin care composition may include applying the skin care composition to relatively clean skin after cleansing the skin. Additionally or alternatively, contacting the skin with the skin care composition may include applying the skin care composition on the face, the neck, or combinations thereof. Additionally or alternatively, contacting the skin with the skin care composition may include applying the skin care composition from a pump container.

Embodiments relate to making the skin care composition, which may include mixing the skin sensory, the skin conditioning agent, and optionally one or more additional ingredients. Traditional methods may be implemented to provide various forms including, without limitation, emulsions, gels, foams, powders, lotions, creams, sprays, mists, milks, or combinations thereof. In one example, embodiments of compositions may be utilized as an anhydrous or hydrous base. Embodiments may further include various packaged formats such as, without limitation, pump containers, spray containers, mist containers, tubes, jars, and the like. Thus, embodiments relate to a packaged consumer product including a skin care composition of any of the preceding embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %) based on a total weight of the composition unless otherwise indicated. Where appropriate, the International Nomenclature of Cosmetic Ingredients (INCI) name of ingredients/components is provided. Any numerical range recited herein is intended to include all sub-ranges subsumed therein, and such ranges are understood to include each and every number and/or fraction between the stated range lower and upper values. Moreover, the term “about” and may refer to deviations of ±20% unless otherwise stated.

Skin Sensory Enhancer

Skin sensory enhancers provide skin benefits such as, for example, anti-aging, skin lightening, skin darkening, exfoliation, anti-acne, and so on. Preferably, a skin care composition includes bakuchiol as a useful skin sensory enhancer to, for example, improve skin tone, improve skin elasticity, smooth fine lines, smooth wrinkles, or combinations thereof. Bakuchiol (e.g., CAS No. 10309-37-2 having the formula C₁₈H₂₄O and molecular weight of 256.38 g/mol), is a terpenophenolic phytoestrogen mainly found in the seeds of Psoralea corylifolia although Bakuchiol is available from a variety of plants such as P. grandulosa, P. drupaceae, Ulmus davidiana, Otholobium pubescens, Piper longum, and Aerva sangulnolenta Blum. Bakuchiol is readily commercially available (e.g., Sigma-Aldrich).

Bakuchiol may be present in an amount of about 0.25 wt. % to about 5.0 wt. %, based on the total weight of a skin care composition. Bakuchiol may, for example, be present in an amount of 0.2375 wt. %, 1.0 wt. %, 1.5 wt. %, 2.0 wt. %, 2.5 wt. %, 3.0 wt. %, 3.5 wt. %, 4.0 wt. %, 4.5 wt. %, 5.1 wt. %, including every number and/or fraction therebetween, based on the total weight of the skin care composition. For example, bakuchiol may be present in an amount greater than 0.5 wt. % and less than or equal to 3.0 wt. %, preferably less than or equal to 1.5 wt. %, based on the total weight of the skin care composition.

In one example, the skin sensory enhancer consists of only bakuchiol. Additionally or alternatively, bakuchiol may be used an alternative to retinol in the skin care composition. Additionally or alternatively, bakuchiol may be present with one or more other ingredients that provide skin benefits such as, for example, vitamins or derivatives thereof, extracts or active molecules thereof, amino acids or derivatives thereof, etc., or combinations thereof. For example, such sensory enhancers may be present from 0.0001 to 20.0 wt. %.

Skin Conditioning Agent

Skin conditioning agents provide skin-conditioning functionality. Examples of useful skin conditioning agents in embodiments include esters, polyols, oils, fatty acids, silicones, salicylates, or combinations thereof. For example, a skin conditioning agent may include isopropyl myristate, isopropyl palmitate, 1-decene polymer (hydrogenated), esters of benzoic acid (e.g., lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate), glycerin, butylene glycol, hexylene glycol, 2,3-butanediol, 1,2-alkane diols (e.g., propylene glycol, 1,2-butanediol, pentylene glycol, ethyl hexanediol, caprylyl glycol, decylene glycol), nut oil, seed oil, fruit oil, lanolin, petrolatum, mineral oil, triglycerides (e.g., caprylic/capric triglyceride), cyclomethicone, dimethicone, adipates, butyloctyl salicylate, C₁₂-C₁₅ alkyl salicylate, capryloyl salicylic acid, hexydodecyl salicylate, isocetyl salicylate, isodecyl salicylate, tridecyl salicylate, or combinations thereof.

Preferably, the skin conditioning agent includes an oil-based agent selected from hemp oil, bisabolol, Adansonia digitata seed oil, or combinations thereof. Cannabis sativa seed oil may be derived from the stalks and seeds of Hemp. Bisabolol, a natural monocyclic sesquiterpene alcohol that is a relatively colorless viscous oil, may be the primary constituent of the essential oil Matricaria recutita and Myoporum crassifolium. Adansonia digitata (Baobab) seed oil may extracted from the seeds of the fruit of the Adansonia digitata tree (Baobab tree). In one example, a ratio of hemp oil, bisabolol, and Adansonia digitata seed oil may be from 1.0:0.1:0.1 to 1.0:3.0:3.0 such as, for example, 1.0:2.4:1.0. The ratios may be the same or different when, for example, one or more of these ingredients are omitted. Thus, for example, a ratio of hemp oil and Adansonia digitata seed oil may be from 1.0:0.1 to 1.0:3.0.

Additionally or alternatively, a skin conditioning agent may include an alcohol-based agent selected from propylene glycol, glycerin, or combinations thereof. Additionally or alternatively, a skin conditioning agent may include a fatty acid-based agent selected from squalane, coco-caprylate/caprate, or combinations thereof. Squalane may refer to a lipid that forms a barrier to traps water in skin. Meanwhile, coco-caprylate/caprate may be an ester from coconut-derived fatty alcohol with caprylic and capric acids from coconut. Additionally or alternatively, as discussed above, other emollients or humectants such as esters, adipates, salicylates, and/or organosilicon compounds, (e.g., silicone fluids, etc.) may be useful.

Skin-conditioning agents may be present from about 0.1 wt. % to about 99.8 wt. %, based on the total weight of the skin care composition. In one example, a total amount of skin-conditioning agent may be present in an amount from about 15.0 wt. % to about 30.0 wt. %, including every number and/or fraction therebetween, based on the total weight of the skin care composition. A skin-conditioning agent may, for example, be present in an amount of 0.08 wt. %, 0.25 wt. %, 0.5 wt. %, 1.0 wt. %, 2.5 wt. %, 5.0 wt. %, 10.0 wt. %, 20.0 wt. %, 40.0 wt. %, 60.0 wt. %, 90.0 wt. %, 99.8 wt. %, including every number and/or fraction therebetween, based on the total weight of the skin care composition. For example, Cannabis sativa seed oil may be present in an amount of 0.1 wt. % to 3.0 wt. %, including every number and/or fraction therebetween, such as 0.5 wt. % or more to 1.5 wt. % or less, based on the total weight of the skin care composition. Thus, in one example, a ratio of Cannabis sativa seed oil to a total of other skin-conditioning agents may be from 1:10 to 1:60.

Surfactant

Surfactants useful in embodiments may include anionic, cationic, nonionic, amphoteric, or combinations thereof. Examples of useful anionic surfactants include carboxylates, amino acid derivatives, alkyl sulphates, alkyl ether sulfates, sulphonates, isethionates, taurates, sulfosuccinates, alkyl sulfoacetates, phosphates, alkyl phosphates, or combinations thereof. Examples of useful cationic surfactants include alkylamines, alkylimidazolines, quaternary ammonium compounds (QAC), or combinations thereof. Examples of useful nonionic surfactants include alkanolamides, amine oxides, esterified carboxylic acids, ethoxylated alcohols, poloxamers, sorbitan esters, or combinations thereof. Examples of useful amphoteric/zwitterionic surfactants include betaines, sultaines, cocamphoacetates, or combinations thereof. Examples of emulsifiers, which are surfactants that stabilize emulsions, may further include silicones such as, without limitation, a copolymer of a silicone, an alkoxylated derivative of a silicone, or combinations thereof.

Preferably a surfactant (e.g., non-ionic, emulsifier) may be selected from a sorbitan ester, an ethoxylated alcohol, or combinations thereof. The sorbitan ester may be formed by reacting the polyol, sorbitol, with a fatty acid (e.g., caprylic, coconut oil-derived fatty acids, isostearic, oleic, stearic, isostearic, olive oil-derived fatty acids, etc.). In one example, the sorbitan ester includes sorbitan isostearate, polysorbate (ethoxylated sorbitan esterified with fatty acids), or combinations thereof. Meanwhile, the ethoxylated alcohol may be formed by reacting a fatty alcohol and ethylene oxide resulting in a molecule that consists of the oleophilic, carbon-rich, fatty alcohol and the hydrophilic, polyoxyethylene chain. In one example, the ethoxylated alcohol includes glycereth-2 cocoate. Useful natural emulsifiers include cetearyl alcohol (and) cetearyl glucoside, glyceryl stearate (and) cetearyl alcohol (and) sodium stearoyl lactylate, cetearyl wheat straw glycosides (and) cetearyl alcohol, sorbitan olivate, cetearyl olivate (and) sorbitan olivate, or combinations thereof.

Surfactants may be present from about 0.1 wt. % to about 20.0 wt. %, based on the total weight of the skin care composition. A surfactant may, for example, be present in an amount of 0.008 wt. %, 0.1 wt. %, 0.5 wt. %, 1.0 wt. %, 5.0, 10.0 wt. %, 22.0 wt. %, including every number and/or fraction therebetween, based on the total weight of the skin care composition.

Binder and/or Thickener

Embodiments may also include binders and/or thickeners to enhance the stability of emulsions, create gels, and/or provide texture. Examples of useful binders and/or thickeners include plant-based (e.g., xanthan gum, guar gum, acacia gum, cellulose derivatives such as hydroxyethyl cellulose, hyaluronic acid, etc.), mineral (e.g., silica, bentonite, magnesium aluminum silicate, etc.), lipid (e.g., dimer dilinoleyl/dimer dilinoleate, cetyl alcohol, stearyl alcohol, waxes such as carnauba wax, stearic acid, etc.), synthetic (e.g., hydroxyethyl acrylate/sodium acryloyldimethyl taurate copolymer, carbomer, acrylates/C₁₀₋₃₀ alkyl acrylate crosspolymer, etc.), or combinations thereof. Binders and/or thickeners may be present from about 0.1 wt. % to about 20.0 wt. %, based on the total weight of the skin care composition. A binder and/or thickener may, for example, be present in an amount of 0.008 wt. %, 0.1 wt. %, 0.5 wt. %, 1.0 wt. %, 5.0, 10.0 wt. %, 22.0 wt. %, including every number and/or fraction therebetween, based on the total weight of the skin care composition.

Film Former

A skin care composition may include a film former to provide a substantially homogenous and/or structured hydro-lipidic film on skin owing to its viscosity-building properties. Generally, a film former may be synthesized from two or more different monomers to form copolymers and/or from individual polymer chains that are connected by bridging molecules or crosslinking agents to form crosslinked copolymers known as crosspolymers. Moreover, a film former may be present in a non-aqueous solvent such as, for example, an alcohol, an ether, a fatty acid, a silicone fluid, an ester, a hydrocarbon, a fluorinated hydrocarbon, or combinations thereof. In one example, a film former is dissolved in isododecane, isohexadacane, diisopropyl adipate, isoparrafins (e.g., C₁₃-C₁₆ isoparrafins), alcohol (e.g., C₂-C₁₀ alcohols, etc.), organosilicon compounds (e.g., disiloxane, dimethicone, cyclopentasiloxane, etc.), or combinations thereof.

A film former may be selected from monomers, copolymers, cross polymers, and/or terpolymers of an organo-silicone hybrid, biopolymer, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. A film former may include, without limitation, crotonic acid/vinyl C₈-C₁₂ isoalkyl esters/VA/bis-vinyldimethicone crosspolymer, acrylate/dimethicone copolymer, VA/butyl maleate/isobornyl acrylate copolymer, methyl dihydroabietate, trimethylsiloxysilicate, acrylate/C₁₂-C₂₂ (meth)acrylate copolymer, C₂₄C₂₈ alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/Dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, shellac, stearyl/octyldodecyl citrate crosspolymer or combinations thereof.

Film formers may be present in an amount of about 0.1 wt. % to about 5.0 wt. %, based on the total weight of the skin care composition. A film former may, for example, be present in an amount of 0.09 wt. %, 0.9 wt. %, 1.0 wt. %, 2.0 wt. %, 3.0 wt. %, 4.0 wt. %, 5.0 wt. %, 5.5 wt. %, including every number and/or fraction therebetween, based on the total weight of the skin care composition.

Other Ingredient(s)

A skin care composition may include other ingredients such as, without limitation, added water, cosmetically acceptable carriers (e.g., non-aqueous, waxes, etc.), oils, sterols, amino acids, moisturizers, powders, colorants, pigments, dyes, pH adjusters, fragrances, cosmetic active ingredients, vitamins, preservatives, preservative boosters, foaming agents, chelating agents, cleansers, dispersing agents, stabilizers, essential fatty acids, sphingolipids, self-tanning compounds, fillers, antioxidants, surfactants, minerals, viscosity and/or rheology modifiers, keratolytics, retinoids, hormonal compounds, alpha-keto acids, anti-bacterial agents, anti-fungal agents, anti-microbials, anti-viral agents, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, staining agents, depigmenting agents, hypopigmenting agents, stabilizers, pharmaceutical agents, sunscreen agents, photostabilizing agents, spherical powders, extracts (fruit, flower, plant), absorbents, salicylic acid, alpha and beta hydroxy acids, retinol and its derivatives, or combinations thereof.

For example, water may be intentionally added in a skin care composition at an amount greater than 15.0 wt. % such as, for example, 25.0 wt. %, 45.0 wt. %, 55.0 wt. %, 65.0 wt. %, 75.0 wt. %, including every number and/or fraction therebetween, based on the total weight of the skin care composition. Thus, an emulsion may have one of an aqueous or oil dispersed phase stabilized by an emulsifier and disposed in the other of the aqueous or oil continuous phase. Water may also be intentionally added in an amount of 15.0 wt. % or less, based on a total weight of the skin care composition, to form a substantially anhydrous skin care composition. However, one or more ingredients may have associated with it absorbed and/or adsorbed water that can be carried into the otherwise substantially anhydrous skin care composition. For economic, sourcing, and/or formulation reasons, it may be disadvantageous to process hygroscopic materials to make them completely free of water. Thus, a substantially anhydrous skin care composition may include 15.0 wt. % or less, preferably 10.0 wt. % or less, of water. For example, a skin care composition may include 5.0 wt. % or less (e.g., s 1.0 wt. %), of water based on a total weight of the skin care composition. In one example, the anhydrous skin care composition is completely free of water. Additionally or alternatively, non-aqueous ingredients that may be used include, for example, an alcohol, a wax, an ether, a fatty acid, a silicone fluid, an ester, a hydrocarbon, or combinations thereof.

Other ingredients may be present in an amount of about 0.01 wt. % to about 99.8 wt. %, based on the total weight of the skin care composition. One or more other ingredients may, for example, be present in an amount of 0.008 wt. %, 0.01 wt. %, 0.1 wt. %, 0.25 wt. %, 0.5 wt. %, 1.0 wt. %, 2.5 wt. %, 5.0 wt. %, 10.0 wt. %, 20.0 wt. %, 40.0 wt. %, 60.0 wt. %, 90.0 wt. %, 99.8 wt. %, including every number and/or fraction therebetween, based on the total weight of the skin care composition.

Product Format(s)

A topical skin care composition may include one or more formulation formats such as, without limitation, an emulsion (O/W, W/O, O/O, multiple emulsion, etc.), a gel, a lotion, a cream, a spray, a mist, a stick, a mouse, a foam, an ointment, a milk, a base, a concentrate, a mist, or combinations thereof. Such formats may initially or finally be substantially aqueous or substantially anhydrous. For example, a substantially anhydrous base may be used in an aqueous emulsion to form a lotion or a cream. Similarly, ingredients and use levels to make such formats may be readily adjusted and mixed in a single phase or in multiple phases to provide a desired commercial sunscreen product. Thus, for example, water content, alcohol content, oil content, emulsifiers, foaming agents, gelling agents, etc., may be included, omitted, adjusted, or mixed in a single phase or in multiple phases to provide a commercial sunscreen product that accounts for cost, aesthetics, and/or product performance objectives.

Moreover, a topical skin care composition can be filled into any number of bottles, tottles, tubes, packets, etc., having a variety of closing structures, pumps, sprays (i.e., aerosols, bag on valve), amongst others, or combinations thereof. A topical skin care composition may, for example, be formulated as a concentrate with a propellant to provide a continuous aerosol spray commercial product to be applied via a spray nozzle. A bag-on-valve system may also be used to deliver the sunscreen composition using compressed gas such as air, nitrogen, or carbon dioxide. It is understood that similar product formats to those discussed in detail herein fall within the true scope and spirit of the present disclosure for any desired commercial product.

Example Skin Care Compositions

Example Table 1 shows skin care compositions, having bakuchiol and hemp oil, that exhibit excellent skin sensory enhancement and/or moisturization with minimized irritation.

EXAMPLE TABLE 1

| | | Approx. |
|---|---|---|
| Component (INCI) | Function | wt. % |
| Example Main Ingredients | | |
| Bakuchiol | Skin Sensory | 0.25-5.0 |
| | Enhancer | |
| Cannabis Sativa Seed Oil | Skin | 0.1-3.0 |
| | Conditioning | |
| | Agent | |
| Example Additional Ingredients | | |
| Aqua (Water), Propanediol, Glycerin, | Solvent, | Q.S. to 100 |
| Coco-Caprylate/Caprate, Dimer Dilinoleyl | Additional Skin | |
| Dimer Dilinoleate, Silica, Hydroxyethyl | Conditioning | |
| Acrylate/Sodium Acryloyldimethyl | Agent, | |
| Taurate Copolymer, Bisabolol, Squalane, | Emulsifier, | |
| Adansonia Digitata Seed Oil, Lavandula | Binder/Thicker, | |
| Angustifolia (Lavender) Oil, Zingiber | Extract, | |
| Officinale (Ginger) Root Extract, | Preservative, | |
| Phenoxyethanol, Polysorbate 60, | pH Adjuster, | |
| Sodium Phytate, Sorbitan Isostearate, | Chelating | |
| Ethylhexylglycerin, Benzoic Acid, | Agent, | |
| Glycereth-2 Cocoate, Potassium | Fragrance | |
| Hydroxide, Linalool | | |

A consumer perception study was conducted over eight weeks with thirty-two subjects for a skin care composition having the ingredients at the corresponding weight percents shown in Example Table 1. The ingredients of the skin care composition were mixed in phases using traditional techniques to form an emulsion. Fine line baseline values and changed values were documented for each subject by a clinical grader based on a point scale. A clinical evaluator also documented objective symptoms of irritation on the face based on a point scale. Questionnaires were provided to the thirty-two subjects for qualitative evaluations.

Example Tables 2-3 show results of clinical assessments for fine lines while Example Tables 4-5 show results of clinical assessments for irritation. While not illustrated, erythema frequency scores from the clinical assessments showed that twenty-seven of twenty-eight subjects with the best possible score for baseline continued to have the best score at eight weeks. None of the remaining subjects had the worst possible score at eight weeks.

As shown in Example Table 2, a marked improvement of 11.44% was observed for fine lines relative to baseline at four weeks and further improved to 22.88% at eight weeks.

| | EXAMPLE TABLE 2 | | |
|---|---|---|---|
| | Clinical Assessment of Fine Lines | | |
| Descriptive Statistics | Baseline | Week 4 | Week 8 |
| Mean | 4.23 | 3.75 | 3.27 |
| SD | 0.99 | 1.12 | 1.20 |
| Median | 4.00 | 3.50 | 3.00 |
| N | 32 | 32 | 32 |
| Percent Change | | −11.44% | −22.88% |

As shown in Example Table 3, there was statistically significant intra-subject change in fine lines, calculated as the difference in values at two different time points. P-values were determined by a Wilcoxon Signed-Rank test with bolded values being significant at P<0.05.

EXAMPLE TABLE 3

Intra-Subject Change in Fine Lines

| | Week 4 Minus | Week 8 Minus |
|---|---|---|
| Descriptive Statistics | Baseline | Baseline |
| Mean | −0.48 | −0.97 |
| SD | 0.53 | 0.59 |
| Median | −0.50 | −1.00 |
| N | 32 | 32 |
| P-Value | 0.0001 | <0.0001 |

As shown in Example Table 4, a marked change of 50.00% was observed for dryness relative to baseline at four weeks and was maintained at eight weeks.

| | EXAMPLE TABLE 4 | | |
|---|---|---|---|
| | Objective Tolerance of Dryness | | |
| Descriptive Statistics | Baseline | Week 4 | Week 8 |
| Mean | 0.13 | 0.06 | 0.06 |
| SD | 0.42 | 0.25 | 0.25 |
| Median | 0.00 | 0.00 | 0.00 |
| N | 32 | 32 | 32 |
| Percent Change | | −50.00% | −50.00% |

As shown in Example Table 5, no edema was observed at eight weeks.

| | EXAMPLE TABLE 5 | | |
|---|---|---|---|
| | Objective Tolerance of Edema | | |
| Descriptive Statistics | Baseline | Week 4 | Week 8 |
| Mean | 0.00 | 0.00 | 0.00 |
| SD | 0.00 | 0.00 | 0.00 |
| Median | 0.00 | 0.00 | 0.00 |
| N | 32 | 32 | 32 |
| Percent Change | | 0 | 0 |

Moreover, 91% of subjects reported improved smoothness and overall feel of skin, 91% of subjects reported improved overnight hydration, 88% of subjects reported brighter-looking skin, 84% of subjects said skin felt firmer, 81% of subjects reported improved overall appearance of skin, 78% of subjects said the product improved the overall texture of their skin, 75% of subjects saw reduced signs of aging, and 72% of subjects saw reduced appearance of fine lines and wrinkles.

Bakuchiol may be an effective plant-based alternative to retinol that provides skin sensory enhancement (e.g., improve skin tone and elasticity, smooths fine lines and wrinkles, etc.). Moreover, Cannabis sativa seed oil may help prevent moisture loss and/or sooth skin.

### Additional Notes and Examples

Additional examples include a composition, a packaged product including a composition, a method of making a composition, and/or a method of using a composition.

Example 1 may include a skin care composition comprising a skin sensory enhancer including bakuchiol present in an amount of 0.25 wt. % to 5.0 wt. %, based on the total weight of the skin care composition, an oil-based skin conditioning agent including Cannabis sativa seed oil present in an amount of 0.1 wt. % to 3.0 wt. %, based on the total weight of the skin care composition, and one or more additional ingredients.

Example 2 may include the skin care composition of Example 1, wherein the skin care composition is free of retinol.

Example 3 may include the skin care composition of any of Examples 1 to 2, wherein the skin care composition is in the form of an emulsion.

Example 4 may include the skin care composition of any of Examples 1 to 3, wherein the oil-based skin conditioning agent further includes bisabolol, Adansonia digitata seed oil, or combinations thereof.

Example 5 may include the skin care composition of any of Examples 1 to 4, wherein a ratio of Cannabis sativa seed oil, bisabolol, and Adansonia digitata seed oil is from 1.0:0.1:0.1 to 1.0:3.0:3.0.

Example 6 may include the skin care composition of any of Examples 1 to 5, further including an alcohol-based skin conditioning agent, a fatty acid-based conditioning agent, or combinations thereof.

Example 7 may include the skin care composition of any of Examples 1 to 6, wherein the alcohol-based skin conditioning agent includes propylene glycol, glycerin, or combinations thereof.

Example 8 may include the skin care composition of any of Examples 1 to 8, wherein the a fatty acid-based skin conditioning agent includes squalane, coco-caprylate/caprate, or combinations thereof.

Example 9 may include the skin care composition of any of Examples 1 to 9, wherein a total amount of skin-conditioning agent is present in an amount from about 15.0 wt. % to about 30.0 wt. %, based on the total weight of the skin care composition.

Example 10 may include the skin care composition of any of Examples 1 to 9, further including one or more additional ingredients selected from added water, skin conditioning agents, cosmetically acceptable carriers, sterols, amino acids, powders, colorants, pigments, dyes, pH adjusters, fragrances, cosmetic active ingredients, vitamins, preservatives, preservative boosters, foaming agents, chelating agents, cleansers, dispersing agents, binders and/or thickeners, stabilizers, essential fatty acids, sphingolipids, self-tanning compounds, fillers, surfactants, emulsifiers, antioxidants, film formers, minerals, viscosity and/or rheology modifiers, keratolytics, retinoids, hormonal compounds, alpha-keto acids, anti-bacterial agents, anti-fungal agents, anti-viral agents, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, staining agents, depigmenting agents, hypopigmenting agents, sunscreen agents, extracts (fruit, flower, plant), absorbents, salicylic acid, alpha and beta hydroxy acids, or combinations thereof.

Example 11 may include the skin care composition of any of Examples 1 to 10, further including an aerosol-free pump spray container within which the skin care composition is packaged.

Example 12 may include a method to provide skin sensory enhancement comprising contacting skin with the skin care composition of any of Examples 1 to 11.

Example 13 may include the method of Example 12, wherein the skin sensory enhancement includes relatively improved smoothness of skin, improved overall feel of skin, brighter-looking skin, firmer skin, improved overall appearance of skin, improved overall texture of skin, reduced signs of aging, reduced appearance of fine lines, reduced appearance of wrinkles, or combinations thereof.

Example 14 may include the method of any of Examples 12 to 13, wherein contacting skin with the skin care composition further provides the moisturization to the skin.

Example 15 may include the method of any of Examples 12 to 14, wherein the moisturization to the skin includes relatively improved skin hydration.

Example 16 may include the method of any of Examples 12 to 15, wherein the method includes applying the skin care composition to the skin at bedtime and leaving the skin care composition disposed on the skin for a predetermined period of time (e.g., an hour, while asleep, etc.).

Example 17 may include the method of any of Examples 12 to 16, wherein the method includes applying the skin care composition to relatively clean skin after an operation using a soap product, a cleanser product, an exfoliation product, or combinations thereof.

Example 18 may include the method of any of Examples 12 to 17, wherein the method includes applying the skin care composition on the face, the neck, or combinations thereof.

Example 19 may include the method of any of Examples 12 to 18, wherein the method includes applying the skin care composition from an aerosol-free pump spray container.

Example 20 may include a method to make a skin care composition of any of Examples 1 to 11.

Example 21 may include the method of Example 20, wherein the method comprises mixing a skin sensory enhancer including bakuchiol in an amount of 0.25 wt. % to 5.0 wt. %, based on the total weight of the skin care composition, an oil-based skin conditioning agent comprising Cannabis sativa seed oil in an amount of 0.1 wt. % to 3.0 wt. %, based on the total weight of the skin care composition, and one or more additional ingredients.

Example 22 may include the method of any of Examples 20 to 21, wherein the mixing is performed by mixing an aqueous phase and an oil phase to form an emulsion.

Example 23 may include the method of any of Examples 20 to 21, wherein the mixing is performed by mixing the ingredients in a non-aqueous solvent to form an single phase, homogenous, anhydrous topical composition.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

What is claimed is:

1. A skin care composition comprising:
a skin sensory enhancer including bakuchiol present in an amount of 0.25 wt. % to 5.0 wt. %, based on the total weight of the skin care composition;
an oil-based skin conditioning agent including Cannabis sativa seed oil present in an amount of 0.1 wt. % to 3.0 wt. %, based on the total weight of the skin care composition; and
one or more additional ingredients.

2. The skin care composition of claim 1, wherein the skin care composition is free of retinol.

3. The skin care composition of claim 1, wherein the skin care composition is in the form of an emulsion.

4. The skin care composition of claim 1, wherein the oil-based skin conditioning agent further includes bisabolol, Adansonia digitata seed oil, or combinations thereof.

5. The skin care composition of claim 4, wherein a ratio of Cannabis sativa seed oil, bisabolol, and Adansonia digitata seed oil is from 1.0:0.1:0.1 to 1.0:3.0:3.0.

6. The skin care composition of claim 1, further including an alcohol-based skin conditioning agent, a fatty acid-based conditioning agent, or combinations thereof.

7. The skin care composition of claim 6, wherein the alcohol-based skin conditioning agent includes propylene glycol, glycerin, or combinations thereof.

8. The skin care composition of claim 6, wherein the a fatty acid-based skin conditioning agent includes squalane, coco-caprylate/caprate, or combinations thereof.

9. The skin care composition of claim 1, wherein a total amount of skin-conditioning agent is present in an amount from about 15.0 wt. % to about 30.0 wt. %, based on the total weight of the skin care composition.

10. The skin care composition of 1, further including added water, one or more additional carriers, sterols, amino acids, powders, colorants, pigments, dyes, pH adjusters, fragrances, cosmetic active ingredients, vitamins, preservatives, preservative boosters, foaming agents, chelating agents, cleansers, dispersing agents, binders and/or thickeners, stabilizers, essential fatty acids, sphingolipids, self-tanning compounds, fillers, surfactants, emulsifiers, antioxidants, film formers, minerals, viscosity and/or rheology modifiers, keratolytics, retinoids, hormonal compounds, alpha-keto acids, anti-bacterial agents, anti-fungal agents, anti-viral agents, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, staining agents, depigmenting agents, hypopigmenting agents, sunscreen agents, extracts (fruit, flower, plant), absorbents, salicylic acid, alpha and beta hydroxy acids, or combinations thereof.

11. The skin care composition of claim 1, wherein the skin care product is packaged in an aerosol-free pump spray container.

12. A method to provide skin sensory enhancement comprising contacting skin with the skin care composition of claim 1.

13. The method to provide skin sensory enhancement of claim 12, wherein the skin sensory enhancement includes relatively improved smoothness of skin, improved overall feel of skin, brighter-looking skin, firmer skin, improved overall appearance of skin, improved overall texture of skin, reduced signs of aging, reduced appearance of fine lines, reduced appearance of wrinkles, or combinations thereof.

14. The method to provide skin sensory enhancement of claim 12, wherein contacting skin with the skin care composition further provides the moisturization to the skin.

15. The method to provide the skin sensory enhancement of claim 14, wherein the moisturization to the skin includes relatively improved skin hydration.

16. The method to provide skin sensory enhancement of any claim 12, wherein the method includes applying the skin care composition to the skin at bedtime and leaving the skin care composition disposed on the skin for a predetermined period of time.

17. The method to provide skin sensory enhancement of claim 12, wherein the method includes applying the skin care composition to relatively clean skin after an operation using a soap product, a cleanser product, an exfoliation product, or combinations thereof.

18. The method to provide skin sensory enhancement of claim 12, wherein the method includes applying the skin care composition on the face, the neck, or combinations thereof.

19. The method to provide skin sensory enhancement of claim 12, wherein the method includes applying the skin care composition from an aerosol-free pump spray container.

20. A method to make a skin care composition comprising mixing:
a skin sensory enhancer including bakuchiol in an amount of 0.25 wt. % to 5.0 wt. %, based on the total weight of the skin care composition;
an oil-based skin conditioning agent comprising Cannabis sativa seed oil in an amount of 0.1 wt. % to 3.0 wt. %, based on the total weight of the skin care composition; and
one or more additional ingredients.
